(19) United States
(12) Patent Application Publication
(10) Pub. No.: US 2009/0291908 A1
(43) Pub. Date: Nov. 26, 2009

(54) Use of thiophoshonoformic acid and nrtis to treat viral infections

(75) Inventors: Shani Waninger, Carlsbad, CA (US); Joan Robbins, Woodland, CA (US); Ramos Silvestri, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Adventrix Pharmaceuticals, Inc., San Diego, CA (US)

(21) Appl. No.: 11/659,068

(22) PCT Filed: Mar. 6, 2006
(86) PCT No.: PCT/US2006/008728

§ 371 Date: Feb. 6, 2009

Related U.S. Application Data
(60) Provisional application No. 60/659,136, filed on Mar. 7, 2005.

(51) Int. Cl. A61K 31/662; A61K 31/7072; A61K 31/708; A61K 31/675; A61P 31/18; A61P 31/12

(52) U.S. Cl. 514/45; 514/120; 514/50; 514/81

(57) ABSTRACT

This invention provides for a method of synergistically reducing viral load in a patient infected with a virus. The method comprises the oral co-administering an amount of thiophosphonoformic acid and an amount of a nucleoside or nucleotide reverse transcriptase inhibitor in a synergistic combination.

### CROSS-REFERENCES TO RELATED APPLICATIONS

This application claims the benefit of provisional application No. 60/659,136 filed on Mar. 7, 2005, the entire contents of which is hereby incorporated herein by reference.

### BACKGROUND OF THE INVENTION

NRTIs, including nucleoside reverse transcriptase inhibitors (NsRTIs) and nucleotide reverse transcriptase inhibitors (NtRTIs) are currently administered in treatments against viral infections, including infections of human immunodeficiency virus (HIV) and hepatitis virus (HBV, HCV). Unfortunately, viral resistance often develops against this class of drugs. See, Daar and Richman, AIDS Res Hum Retroviruses, (2005) 21:343-57. Further, various side effects of NRTIs are harmful to the patient, and can discourage patient compliance, diminishing the effectiveness of this class of drugs. See, for example, Raines, et al., J Assoc Nurses AIDS Care (2005) 16:35; Cherry, et al., Sex Health (2005) 2:1; Abrescia, et al., Curr Pharm Des. (2005) 11:3697.

Thiophosphonoformic acid (“Thiovir™”) is a sulfur-containing oral replacement for foscaret, a highly effective, broad-spectrum antiviral with limited usage due to toxicity and low bioavailability. An oral form of thiophosphonoformic acid (Thiovir™) under development by the Assignee hereof provides improved efficacy and bioavailability making it an attractive clinical candidate.

It has been suggested to combine thiophosphonoformic acid with other antiviral compounds such as zidovudine (AZT). See, U.S. Pat. No. 5,183,812 (column 21). However, the synergistic antiviral effects achieved by combining thiophosphonoformic acid with one or more NRTIs, which would allow for reducing the efficacious dosages of one or both of these active agents, has not been recognized. There exists a need to improve the effectiveness of thiophosphonoformic acid and NRTIs by preventing or delaying viral resistance, and by decreasing dosages to reduce toxicities and unwanted side effects while still maintaining efficacy in reducing viral load in a patient.

The present invention addresses this and other needs.

### BRIEF SUMMARY OF THE INVENTION

The present invention provides methods and compositions for co-delivery to an individual in need thereof of a combination of thiophosphonoformic acid and an NRTI, wherein the active agents are delivered in proportions and amounts such that their combined anti-viral activity is synergistic.

Accordingly, in a first aspect, the invention provides methods of reducing viral load or viral titer in a patient infected with a virus, the method comprising the oral co-administering to the patient of an amount of thiophosphonoformic acid and an amount of one or more of a nucleoside or nucleotide reverse transcriptase inhibitor, or mixtures thereof, where the amounts being co-administered are sufficient to synergistically lower the viral load or viral titer and where the thiophosphonoformic acid is orally administered in a dosage range of between 2 mg/kg to 50 mg/kg per day.

In another aspect, the invention provides methods of preventing or delaying the resistance of a virus to an NRTI, the method comprising the oral co-administering to the patient of an amount of thiophosphonoformic acid and an amount of one or more of a nucleoside or nucleotide reverse transcriptase inhibitor, or mixtures thereof, where the amounts being co-administered are sufficient to synergistically lower the viral load or viral titer and where the thiophosphonoformic acid is orally administered in a dosage range of between 2 mg/kg to 50 mg/kg per day.

The thiophosphonoformic acid can be efficaciously administered according to the present methods and in the present compositions at dosages that are between about 10-fold and 100-fold smaller in comparison to dosages when thiophosphonoformic acid is administered without an NRTI. In some embodiments, the thiophosphonoformic acid is administered according to the present invention at dosages that are 200-fold, or 300-fold smaller in comparison to dosages when thiophosphonoformic acid is administered without an NRTI. In some embodiments, the thiophosphonoformic acid is administered in a dosage range of between 2 mg/kg to 50 mg/kg per day. In some embodiments, the thiophosphonoformic acid is administered in a dosage range of between 15 mg/kg to 40 mg/kg or 20 mg/kg to 35 mg/kg per day. In some embodiments, the thiophosphonoformic acid is administered at a dosage of about 5, 10, 15, 20, 25, 30, 35, 40, 45, 50 mg/kg per day.

When co-administered with thiophosphonoformic acid, the NRTI can be efficaciously administered at dosages that that are about 2-fold, 5-fold, 10-fold, 20-fold, 50-fold, 100-fold smaller in comparison to dosages when the NRTI is administered without thiophosphonoformic acid.

In some embodiments, a nucleoside reverse transcriptase inhibitor is co-administered. The nucleoside reverse transcriptase inhibitor can be, for example, zidovudine, didanosine, stavudine, lamivudine, abacavir, emtricitabine, zalcitabine, dexelvucitabine, alovudine, amdoxovir, elvucitabine, AVX754, BCH-189, phosphazid, racivir, SP1093V, stampidine, or mixtures thereof. In some embodiments, the nucleoside reverse transcriptase inhibitor is zidovudine.

In some embodiments, a nucleotide reverse transcriptase inhibitor is co-administered. In some embodiments, the nucleotide reverse transcriptase inhibitor is tenofovir, adefovir, or mixtures thereof.

In some embodiments, the virus is a retrovirus. The retrovirus can be a lentivirus. The lentivirus can be, for example, HIV (human immunodeficiency virus), BIV (bovine immunodeficiency virus), CAEV (caprine encephalitis-arthritis virus), EIAV (equine infectious anemia virus), FIV (feline immunodeficiency virus), GLV (goat leukoencephalitis virus), JDV (Jembrana), MVV (maedi/visna virus), PPV progressive pneumonia virus), SIV (simian immunodeficiency virus). In some embodiments, the lentivirus is a T-cell lymphotropic virus, for example, a Human Immunodeficiency Virus (HIV). The HIV can be type 1 (HIV-1) or type 2 (HIV-2). The HIV can be from any HIV clade (e.g., A-G), strain or variant, including, for example, HIV-1:ARV-2/SF-2; HIV-1:BRU (LAI); HIV-1:CAM1; HIV-1:ELI; HIV-1:HXB2; HIV-1:IIIB; HIV-1:MAL; HIV-1:MN; HIV-1:NDK; HIV-1:PV22; HIV-1:RF; HIV-1:U455; HIV-1:Z2.

In some embodiments, the virus is a herpes virus. The herpes virus can be a Alphaheipesvirus, for example, a Sitnplexvirus (e.g., human herpesvirus 1) or a Varicellovirus (e.g., human herpesvirus 3 or Varicella-Zoster virus 1 (VSV1)). The herpes virus can be a Betaherpesvirus, for example, a Cytomegalovirus (e.g., human herpesvirus 5) or a Roseolovirus (e.g., human herpesvirus 6). In some embodiments, the herpes virus is a Gammaherpesvirus, for example, a Lymphociyptovirus (e.g. human herpesvirus 4 or Epstein-Barr Virus (EBV)) or a Rhadinovirus (e.g., Ateline herpesvirus 2).

As appropriate, the thiophosphonoformic acid and the NRTI can be simultaneously or sequentially co-administered. In some embodiments, the thiophosphonoformic acid and the NRTI are simultaneously administered in a combined formulation.

The thiophosphonoformic acid and the NRTI independently can be orally or parenterally administered. In some embodiments, at least one of the thiophosphonoformic acid and the NRTI is administered orally. In some embodiments, at least one of the thiophosphonoformic acid and the NRTI is administered parenterally, for example, intraveneously. Oral formulations can be solid (e.g., tablets, capsules, powders) or liquid (e.g., syrup). In some embodiments, thiophosphonoformic acid and the nucleoside or nucleotide reverse transcriptase inhibitor are administered concurrently in a combined oral formulation.

The invention further provides for an oral medicament for lowering viral load or viral titer in a patient infected with a virus, where the medicament is a combined formulation comprising an amount of thiophosphonoformic acid and an amount of an NRTI where the thiophosphonoformic acid and NRTI are present in amounts sufficient to synergistically lower viral load or viral titer and the medicament contains between 150 mg and 750 mg of thiophosphonoformic acid per combined formulation.

The invention further provides the use of thiophosphonoformic acid and a nucleoside or nucleotide reverse transcriptase inhibitor in the manufacture of a medicament for the therapeutic and/or prophylactic treatment of a viral infection where the thiophosphonoformic acid and nucleoside or nucleotide reverse transcriptase inhibitor are present in amounts sufficient to synergistically lower viral load or viral titer and the medicament contains between 150 mg and 750 mg of thiophosphonoformic acid per combined formulation.

The invention further provides the use of thiophosphonoformic acid and a nucleoside or nucleotide reverse transcriptase inhibitor in the preparation of an anti-viral agent in ready-to-use drug form for treating or preventing virus infections where the where the thiophosphonoformic acid and nucleoside or nucleotide reverse transcriptase inhibitor are present in amounts sufficient to synergistically lower viral load or viral titer and the ready-to-use drug form contains between 150 mg and 750 mg of thiophosphonoformic acid per form.

The preferred embodiments for the compositions and uses are the same as described above.

### BRIEF DESCRIPTION OF THE DRAWINGS

FIG. 1 illustrates a panel of NRTI and NNRTI resistant HIV-1. NRTI and NNRTI susceptibility was tested by PhenoSense™ reporter assay as described in the Examples. Numbers representing fold change ranges of drug susceptibility are as follows: 1: >2.5 fold, 2: 2.5 to 10 fold, 3: 10 to 50 fold and 4: >50 fold. Right Panel: specific mutations in each variant virus which confer resistance to NRTI and NNRTI.

FIG. 2 illustrates that the panel of NRTI and NNRTI resistant HIV-1 virus of FIG. 1 are sensitive to foscarnet and Thiovir™. Left Panel: Identical to that of FIG. 1. Right Panel Susceptibility of resistant virus to stavudine, zidovudine, efavirenz, foscarnet, nevirapine and Thiovir™. IC₅₀ was determined by PhenoSense™ reporter assay. Fold Change (PC) was determined by IC₅₀patient/IC₅₀reference. The reference IC₅₀ is that of a wild type standard virus. S/R indicates whether a virus is considered sensitive (S) or resistant (R) to a drug. Clinical cutoff, the level at which probability of failure significantly increases, is highlighted in light grey and hypersensitivity in dark grey.

### DETAILED DESCRIPTION OF THE INVENTION

### 1. Introduction

Thiophosphonoformic acid (TPFA) is a sulfur analogue of the broad-spectrum anti-viral therapeutic foscarnet (phosphonoformic acid; PFA). Foscarnet, a non-nucleoside reverse transciptase inhibitor (NNRTI), is a pyrophosphate analogue with a unique mode of action among NNRTI drugs. Foscarnet inhibits pyrophosphate exchange by viral DNA polymerases through competitive binding for the pyrophosphate enzyme binding site. Foscarnet and its analogues, such as Thiovir™, inhibit DNA chain elongation catalyzed by viral DNA polymerases leading to inhibition of viral replication of both DNA and RNA viruses, including HIV.

In vitro anti-HIV efficacy studies indicate Thiovir™ has approximately equal antiviral activity to foscarnet. Furthermore, Thiovir™ has antiviral activity against several complex NRTI and NNRTI resistant strains. All HIV-1 variants examined were sensitive to Thiovir™ and foscarnet. In most case, HIV-1 variants were hypersensitive to Thiovir™, as compared to wild-type virus.

Efficacy studies examining combination therapy of Thiovir™ plus NsRTI or NtRTI drugs show synergistic anti-viral activity. Therefore, the dosage of Thiovir™ can be decreased allowing for suitable drug regimens combining Thiovir™ with nucleoside and nucleotide reverse transcriptase inhibitors. For example, Thiovir™ combined with zidovudine (AZT) results in strong synergistic activity while combinations of foscarnet and zidovudine exhibit only mild synergistic to antagonistic behavior.

The resistance profile of Thiovir™, like foscarnet, is unique among NNRTIs reflecting the distinct mechanism of action of this drug class. Virus selected for resistance to Thiovir™ in vitro shows hypersensitivity to AZT. Therefore, Thiovir™ can re-enable use of AZT in AZT resistant patients. Furthermore, all attempts to select for virus co-resistant to both Thiovir™ and AZT were unsuccessful. This suggests that the resistance profiles of Thiovir™ and AZT are mutually exclusive (i.e., they are competitive inhibitors of each other). Together, this data provide favorable support for combination treatment with Thiovir™ and AZT.

### 2. Definitions

The term “retrovirus” refers a class of enveloped viruses of the family Retroviridae (Taxonomy code 61) that have their genetic material in the form of RNA and use the reverse transcriptase enzyme to translate their RNA into DNA in the host cell (see also, Coffin, et al., Retroviruses, (1997) Cold Spring Harbor Laboratory Press). Genuses of retroviruses include Alpharetrovirus, Betaretrovirus, Gammaretrovirus, Deltaretrovirus, Epsilonretrovirus, and Lentivirus. Virus taxonomy is described on the worldwide web at ncbi.nlm.nih.gov/ICTVdb/ICTVdB.

The term “herpes virus” refers to an enveloped DNA virus within the Herpesviridae virus family (Virus Taxonomy Code 31)

The term “viral load” or “viral titer” interchangeably refer to the number of virus particles in a sample of body fluid (e.g., blood, serum, plasma, saliva, urine) from an infected individual.

The term “synergistic” or “synergy” interchangeably refer to the interaction of two or more agents so that their combined effect is greater than the sum of their individual effects. Synergistic drug interactions can be determined using the median effect principle (see, Chou and Talalay (1984) Adv Enzyme Regul 22:27 and Synergism and Antagonism in Chemotherapy, Chou and Rideout, eds., 1996, Academic, pp. 61-102) and quantitatively determined by combination indices using the computer program Calcusyn (Chou and Hayball, 1996, Biosoft, Cambridge, Mass.). See also, Reynolds and Maurer, Chapter 14 in Methods in Molecular in Medicine, vol. 110: Chemosensitivity, Vol. 1: In Vitro Assays, Blumenthal, ed., 2005, Humana Press. Combination indices (CI) quantify synergy, summation and antagonism as follows: CI<1 (synergy); CI=1 (summation); CI>1 (antagonism). A CI value of 0.7-0.9 indicates moderate to slight synergism. A CI value of 0.3-0.7 indicates synergism. A CI value of 0.1-0.3 indicates strong synergism. A CI value of <0.1 indicates very strong synergism.

The phrase “sufficient to synergistically lower the viral load or viral titer” refers an amount and proportion of two or more active agents such that their combined effects lower viral titer or viral load greater than the sum of the individual active agents. The synergistic effect on viral load is measured in vitro or in vivo using test known in the art, including the tests described herein (e.g. MAGI assay, p24 assay). Sufficient amounts and proportions of the two or more active agents to operate synergistically to lower viral load can be determined by the amounts administered or by the concentrations of the active agents in a body fluid (e.g., blood, serum, plasma, saliva, urine).

The term “thiophosphonoformic acid” or “TPFA” or “Thiovir™” interchangeably refer to a thio-analogue of phosphonoformic acid having the following chemical formula (HO)₂P(S)COOH:

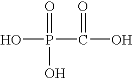

The term “nucleoside reverse transcriptase inhibitor” or “NsRTI” refers to a member of a class of drugs that are chemical analogs of a nucleoside that inhibit a viral reverse transcriptase or polymerase enzyme and therefore the ability of a virus to infect a host cell. A nucleoside reverse transcriptase inhibitor can be a chemical analog of a ribonucleoside or a deoxyribonucleoside.

The term “nucleotide reverse transcriptase inhibitor” or “NtRTI” refers to a member of a class of drugs that are chemical analogs of a nucleotide (a nucleoside 5′-monophosphate) that inhibit a viral reverse transcriptase or polymerase enzyme and therefore the ability of a virus to infect a host cell. A nucleotide reverse transcriptase inhibitor can be a chemical analog of a ribonucleotide or a deoxyribonucleotide.

The term “non-nucleoside reverse transcriptase inhibitor” or “NNTI” refers to a class of drugs that are not chemical analogs of a nucleoside or nucleotide and that allosterically inhibit a viral reverse transcriptase or polymerase enzyme. A NNRTI binds to a reverse transcriptase or a polymerase at a site distinct from the binding site of a NRTI, i.e., the active site of the enzyme.

The term “co-administer” refers to the simultaneous presence of two active agents in the blood of an individual. Active agents that are co-administered can be concurrently or sequentially delivered.

### 3. Compositions

The present invention provides pharmaceutical compositions comprising a mixture of an effective amount of thiophosphonoformic acid and an effective amount of one or more nucleoside/nucleotide reverse transcriptase inhibitors, such that the amounts and proportions of the active agents synergistically lower viral load in an individual.

i. Active Agents

### 1. Thiophosphonoformic Acid

As used herein, thiophosphonoformic acid (TPFA) or “Thiovir™” refers to an active agent having the following chemical formula:

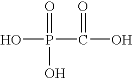

Thiophosphonates, including Thiovir, and methods for their synthesis are disclosed, for example, in U.S. Pat. Nos. 5,072,032; 5,183,812; 6,147,244 and 6,284,909, the entire contents of which are hereby incorporated herein by reference for all purposes. Thiophosphonoformic acid can also be synthesized through commercial service providers, for example, Natland International Corp., Morrisville, N.C.; Custom Synthesis Inc., Delray Beach, Fla.; D Pharm Innovative Biopharmaceuticals, Princeton, N.J. and Chemshop, Weert, The Netherlands.

### 2. Nucleoside Reverse Transcriptase Inhibitors

Any nucleoside reverse transcriptase inhibitors (NsRTI), or mixtures thereof find use in the present methods and compositions. Exemplified NsRTIs include, without limitation, zidovudine (AZT), didanosine, stavudine, lamivudine, abacavir, emtricitabine, zalcitabine, dexelvucitabine, alovudine, amdoxovir, elvucitabine, AVX754, BCH-189, phosphazid, racivir, SP1093V, stampidine, phosphonovir, and combinations thereof. Those of skill in the art will recognize that other analogs of adenosine, guanosine, cytidine, thymine and uridine will function as NsRTIs. Additional NsRTIs of use are disclosed, for example, in U.S. Pat. Nos. 6,514,979; 6,503,890; 6,995,283; and in U.S. Patent Publication Nos. 2004/0235869 and 2004/0127436, the disclosures of which are hereby incorporated herein by reference in their entirety for all purposes.

A nucleoside reverse transcriptase inhibitor can be determined by its function to inhibit the enzymatic activity of a reverse transcriptase (RT). Assays for the determination of the inhibition of a reverse transcriptase are well known in the art. For example, in one assay, reverse transcriptase activity is determined by monitoring the formation of radioactively labeled nuclei acid product absorbed onto ion exchange paper discs in the presence or absence of a candidate RT inhibitor. (See, for example, Canard, et al., (1999) 274:35768; Meyer, et al., (2000) EMBO J. 19:3520; Selmi, et al., (2001) J Biol Chem 276:13965; and Deval, et al, (2004) J Biol Chem 279:25489).

### 3. Nucleotide Reverse Transcriptase Inhibitors

Any nucleotide reverse transcriptase inhibitors (NtRTI), or mixtures thereof, find use in the present methods and compositions. Exemplary NtRTIs include, without limitation, tenofovir, adefovir, and combinations thereof. Those of skill in the art will recognize that other analogs of adenosine 5′-monophosphate (AMP), guanosine 5′-monophosphate (GMP), cytidine 5′-monophosphate (CMP), thymine 5′-monophosphate (dTMP) and uridine 5′-monophosphate (UMP) will function as NtRTIs. Further the NsRTIs discussed above can be in nucleotide form. Additional NtRTIs of use are disclosed, for example, in U.S. Patent Publication No. 2004/0127436, the disclosure of which is hereby incorporated herein by reference in their entirety for all purposes.

A nucleotide reverse transcriptase inhibitor can be determined by its function to inhibit the enzymatic activity of a reverse transcriptase (RT), for example, using the assays described above.

### 4. Other Active Ingredients

Combinations of thiophosphonoformic acid and one or more nucleoside/nucleotide reverse transcriptase inhibitors can be administered with additional active ingredients used in the treatment against viral infection. For example, combinations of thiophosphonoformic acid and one or more nucleoside/nucleotide reverse transcriptase inhibitors can be co-administered with protease inhibitors, or with one or more additional NNRTIs, as a part of highly active antiretroviral therapy (HAART) in the treatment of HIV.

In some embodiments, the pharmaceutical compositions comprise one or more nucleoside reverse transcriptase inhibitors selected from the group consisting of zidovudine (AZT), didanosine, stavudine, lamivudine, abacavir, emtricitabine, zalcitabine, dexelvucitabine, alovudine, amdoxovir, elvucitabine, AVX754, BCH-189, phosphazid, racivir, SP1093V, stampidine, phosphonovir, idoxuridine. In some embodiments, the pharmaceutical compositions comprise one or more nucleotide reverse transcriptase inhibitors selected from the group consisting of tenofovir and adefovir.

A thiophosphonoformic acid and nucleoside/nucleotide reverse transcriptase inhibitor combination of this invention can be incorporated into a variety of formulations for therapeutic administration. More particularly, a combination of the present invention can be formulated into pharmaceutical compositions, together or separately, by formulation with appropriate pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of a thiophosphonoformic acid and nucleoside/nucleotide reverse transcriptase inhibitor combination can be achieved in various ways, including oral, buccal, parenteral, intravenous, intradermal (e.g., subcutaneous, intramuscular), topical, transdermal, etc., administration. Moreover, the compound can be administered in a local rather than systemic manner, for example, in a depot or sustained release formulation. In a preferred embodiment, the invention provides for a pharmaceutical composition comprised of thiophosphonoformic acid and one or more nucleoside/nucleotide reverse transcriptase inhibitors for oral delivery (e.g., a tablet, a capsule, a powder, a liquid, a syrup, etc.).

Suitable formulations for use in the present invention can be found in Remington: The Science and Practice of Pharmacy, 20th and 21st Editions, supra. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

In one preferred embodiment, a thiophosphonoformic acid and nucleoside/nucleotide reverse transcriptase inhibitor combination is prepared for delivery in a sustained-release, controlled release, extended-release, timed-release or delayed-release formulation, for example, in semipermeable matrices of solid hydrophobic polymers containing the therapeutic agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients (see, for example, Huang, et al. Drug Dev. Ind. Pharm. 29:79 (2003); Pearnchob, et al. Drug Dev. Ind. Pharm. 29:925 (2003); Maggi, et al. Eur. J. Pharm. Biopharm. 55:99 (2003); Khanvilkar, et al., Drug Dev. Ind. Pharm. 228:601 (2002); and Schmidt, et al., Int. J. Pharm. 216:9 (2001)). Sustained-release delivery systems can, depending on their design, release the compounds over the course of hours or days, for instance, over 4, 6, 8, 10, 12, 16, 20, 24 hours or more. For example, sustained release formulations can be prepared using naturally-occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

The sustained or extended-release formulations can also be prepared using natural ingredients, such as minerals, including titanium dioxide, silicon dioxide; zinc oxide, and clay (see, U.S. Pat. No. 6,638,521, herein incorporated by reference). Exemplified extended release formulations that can be used in delivering a thiophosphonoformic acid and nucleoside/nucleotide reverse transcriptase inhibitor combination of the present invention include those described in U.S. Pat. Nos. 6,635,680; 6,624,200; 6,613,361; 6,613,358, 6,596,308; 6,589,563; 6,562,375; 6,548,084; 6,541,020; 6,537,579; 6,528,080 and 6,524,621, each of which is hereby incorporated herein by reference. Controlled release formulations of particular interest include those described in U.S. Pat. Nos. 6,607,751; 6,599,529; 6,569,463; 6,565,883; 6,482,440; 6,403,597; 6,319,919; 6,150,354; 6,080,736; 5,672,356; 5,472,704; 5,445,829; 5,312,817 and 5,296,483, each of which is hereby incorporated herein by reference. Those skilled in the art will readily recognize other applicable sustained release formulations.

For oral administration, a thiophosphonoformic acid and nucleoside/nucleotide reverse transcriptase inhibitor combination can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

The compounds can be formulated for parenteral administration by injection, e.g., by bolus injection or continuous infusion. For injection, a thiophosphonoformic acid and nucleoside/nucleotide reverse transcriptase inhibitor combination can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, a combination of the invention can be formulated in aqueous solutions, preferably in physiologically compatible buffers such as Hanks's solution, Ringer's solution, or physiological saline buffer. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

For topical administration, the agents are formulated into ointments, creams, salves, powders and gels.

Systemic administration can also be by transmucosal or transdermal means. For transmucosal or transdermal administration, penetrants appropriate to the barrier to be permeated are used in the formulation. In one embodiment, the transdermal delivery agent can be, for example, DMSO, urea, 1-methyl-2-pyrrolidone, oleic acid, or a terpene (e.g., l-menthol, d-limonene, RS-(+/−)-beta-citronellol, geraniol). Further percutaneous penetration enhancers are described, for example, in Percutaneous Penetration Enhancers, Smith and Maibach, eds., 2^{nd} edition, 2005, CRC Press. Transdermal delivery systems can include, e.g., patches. For transmucosal administration, penetrants appropriate to the barrier to be permeated are used in the formulation. Such penetrants are generally known in the art. Exemplified transdermal delivery formulations that can find use in the present invention include those described in U.S. Pat. Nos. 6,589,549; 6,544,548; 6,517,864; 6,512,010; 6,465,006; 6,379,696; 6,312,717 and 6,310,177, each of which are hereby incorporated herein by reference.

For buccal administration, the compositions can take the form of tablets or lozenges formulated in conventional manner.

In addition to the formulations described previously, a thiophosphonoformic acid and nucleoside/nucleotide reverse transcriptase inhibitor combination of the present invention can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions suitable for use in the present invention include compositions wherein the active ingredients are contained in a therapeutically effective amount. The amount of composition administered will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein. Generally, an efficacious or effective amount of a thiophosphonoformic acid and nucleoside/nucleotide reverse transcriptase inhibitor combination is determined by first administering a low dose of one or both active agents and then incrementally increasing the administered dose or dosages until a desired effect of reduced viral titer is observed in the treated subject, with minimal or no toxic side effects. Applicable methods for determining an appropriate dose and dosing schedule for administration of a combination of the present invention are described, for example, in Goodman and Gilman's The Pharmacological Basis of Therapeutics, 11th Edition., supra, and in Remington: The Science and Practice of Pharmacy, 20th and 21st Editions, supra.

Dosage amount and interval can be adjusted individually to provide plasma levels of the active compounds which are sufficient to maintain therapeutic effect. Preferably, therapeutically effective serum levels will be achieved by administering single daily doses, but efficacious multiple daily dose schedules are included in the invention. In cases of local administration or selective uptake, the effective local concentration of the drug may not be related to plasma concentration. One having skill in the art will be able to optimize therapeutically effective local dosages without undue experimentation.

### 4. Methods

a. Co-Administration of Thiophosphonoformic Acid and a Nucleoside/Nucleotide Reverse Transcriptase Inhibitor

The present methods are directed to the co-administration of thiophosphonoformic acid TPFA) and one or more nucleotide/nucleoside reverse transcriptase inhibitors (NtRTI and/or NsRTI). The thiophosphonoformic acid and the NtRTI and/or NsRTI can be delivered concurrently or sequentially, so long as both the thiophosphonoformic acid and the NtRTI and/or NsRTI are present in the blood.

i. Routes of Administration

As discussed above, thiophosphonoformic acid and one or more nucleoside/nucleotide reverse transcriptase inhibitors can be independently co-administered by any appropriate route of administration. The active ingredients can be administered by the same or different routes of administration, as appropriate. In some embodiments, at least one of the active ingredients is administered orally. In some embodiments, the combination of active ingredients is concurrently orally administered. In some embodiments, at least one of the active ingredients is administered parenterally, for example, intravenously, intramuscularly, subcutaneously, topically, intravaginally, rectally, intranasally, intrathecally, intraocularly.

A combination of thiophosphonoformic acid and one or more nucleoside/nucleotide reverse transcriptase inhibitors can be administered to a subject, e.g., a human patient, a domestic animal (e.g., a cat or a dog), an agricultural animal (e.g., a horse, a cow, a sheep, or a goat), independently or together in the form of their pharmaceutically acceptable salts, or in the form of a pharmaceutical composition where the compounds are mixed with suitable carriers or excipients in a therapeutically effective amount, e.g., at doses effective to synergistically effect desired reduction in viral load or viral titer.

ii. Dosing

As discussed above, those of skill in the art will recognize that that appropriate doses of thiophosphonoformic acid and one or more nucleoside/nucleotide reverse transcriptase inhibitors will depend on several factors, including without limitation, the selected route of administration, the age, weight and prognosis of the patient, the progression of the illness, etc. General considerations for dosing and formulation of thiophosphonoformic acid and one or more nucleoside/nucleotide reverse transcriptase inhibitors, individually or combined, can be found in Goodman and Gilman's the Pharmacological Basis of Therapeutics, Goodman, et al., eds., 11^{th} Edition, 2005, McGraw-Hill and Remington: The Science and Practice of Pharmacy, 20th and 21st Editions, Gennaro and University of the Sciences in Philadelphia, Eds., Lippencott Williams & Wilkins (2003 and 2005).

Thiophosphonoformic acid can be administered in an amount of from about 2 mg/kg to about 50 mg/kg per day, although the doses can be more or less, depending on the route of administration. For example, the doses can be less if the TPFA is administered intravenously. In some embodiments, the thiophosphonoformic acid is administered in an amount of from about 20 mg/kg to about 35 mg/kg per day. In some embodiments, the thiophosphonoformic acid is administered in an amount of about 5, 10, 15, 20, 25, 30, 35, 40, 45, 50 mg/kg per day, or any integer within the range of 2-50 mg/kg per day.

The one or more nucleoside/nucleotide reverse transcriptase inhibitors can be administered in doses according to those approved by FDA, although the doses can be less. Approved doses for nucleoside/nucleotide reverse transcriptase inhibitors can be found, for example, in the FDA Orange Book, available on the worldwide web at fda.gov/cder/ob/default.htm. For example, approved oral doses for approved nucleoside/nucleotide reverse transcriptase inhibitors are as set forth in the table below. Tablets or capsules of nucleoside/nucleotide reverse transcriptase inhibitors can be administered one, two, three, four or five times per day, as appropriate.

Oral Doses of Approved NsRTIs/NtRTIs

| NsRTI/NtRTI | Tablet/Capsule Dose (mg) |
|---|---|
| zalcitabine | 0.375-0.75 |
| stavudine | 15-40 |
| lamivudine | 150 |
| emtricitabine | 200 |
| abacavir tenofovir | 300 |
| zidovudine | 100-300 |
| didanosine | 25-400 |

In some embodiments, at least one of thiophosphonoformic acid and the one or more nucleoside/nucleotide reverse transcriptase inhibitors are administered in subtherapeutic doses of the individual active agents. A subtherapeutic dose refers to an amount of an individual active agent that is insufficient to produce an antiviral effect, as measured in vivo or in vitro, using one of the assays described herein. A subtherapeutic dose also refers to a dose amount that is 80% or less of the smallest reference dose amount of an approved active agent. Reference dose amounts are available to those of skill in the art, for example, in Goodman and Gilman's, supra and in the Physician's Desk Reference, 2006, Thompson Publishing. Without being bound to theory, the synergistic antiviral effects of a combination of thiophosphonoformic acid and one or more nucleoside/nucleotide reverse transcriptase inhibitors can allow for administration of a subtherapeutic dose of at least one of thiophosphonoformic acid and the one or more nucleoside/nucleotide reverse transcriptase inhibitors. Administration of subtherapeutic doses of thiophosphonoformic acid or the nucleoside/nucleotide reverse transcriptase inhibitors individually does not produce a significant antiviral effect. However, administration of a subtherapeutic dose of at least one active agent in a combination of thiophosphonoformic acid and one or more nucleoside/nucleotide reverse transcriptase inhibitors can produce a antiviral effect, with a lower risk of side effects.

Dosages of thiophosphonoformic acid and/or the one or more nucleoside/nucleotide reverse transcriptase inhibitors also can be expressed in term of dose reduction index (DRI). Dose reduction index is a determination of the fold dose reduction allowed for each drug when given in synergistic combination, as compared with the concentration of a single agent that is needed to achieve the same effect level. The synergistic antiviral effects between thiophosphonoformic acid and one or more NRTIs provides for a DRI for thiophosphonoformic acid that is at least about 10, 50, 100, 150, 200, 250, 300, or more. The synergistic antiviral effects between thiophosphonoformic acid and one or more NRTIs provides for a DRI for the one or more NRTIs that is at least about 2, 5, 10, 20, 50, 100, or more.

The combination of thiophosphonoformic acid and one or more nucleoside/nucleotide reverse transcriptase inhibitors are administered in amounts such that the molar ratios of the active ingredients allow for synergistic antiviral effects. The thiophosphonoformic acid can be administered at an equimolar ratio to the one or more NsRTI/NtRTIs (1:1 molar ratio). The thiophosphonoformic acid can be administered at a ten-fold (10:1) or hundred-fold (100:1) greater molar ratio to the one or more NsRTI/NtRTIs, or at any molar ratio from about 1:1 to about 100:1 (TPFA:NRTI), for example, 1:1, 5:1, 10:1, 20:1, 50:1, 100:1.

The combination of thiophosphonoformic acid and one or more nucleoside/nucleotide reverse transcriptase inhibitors are administered in amounts such that the combination index value (CI), determined for example, in a MAGI assay, a PhenoSense™ assay or a p24 antigen capture assay, is less than 1.0. Preferably, the combination of thiophosphonoformic acid and one or more nucleoside/nucleotide reverse transcriptase inhibitors are administered in amounts such that the combination index value is less than about 0.9. More preferably, the combination of thiophosphonoformic acid and one or more nucleoside/nucleotide reverse transcriptase inhibitors are administered in amounts such that the combination index value is less than about 0.6.

iii. Scheduling

The thiophosphonoformic acid and the one or more nucleoside/nucleotide reverse transcriptase inhibitors can be administered concurrently or independently, one, two, three, four or more times in a 24-hour period, as needed. In one embodiment, the thiophosphonoformic acid and one or more nucleoside/nucleotide reverse transcriptase inhibitors are administered concurrently. In one embodiment, the thiophosphonoformic acid and one or more nucleoside/nucleotide reverse transcriptase inhibitors are administered concurrently once daily, for example, in a sustained-release or delayed release formulation. In one embodiment, the thiophosphonoformic acid and one or more nucleoside/nucleotide reverse transcriptase inhibitors are administered concurrently multiple times daily, for example, two, three, or four times daily.

### 5. Assays for Synergistic Activity

The following assays can be used to determine synergistic antiviral activity of combinations of thiophosphonoformic acid and one or more nucleoside/nucleotide reverse transcriptase inhibitors.

a. In vitro

Amounts and proportions of combinations of thiophosphonoformic acid and one or more nucleoside/nucleotide reverse transcriptase inhibitors that synergistically reduce viral titer can be determined using in vitro assays.

i. MAGI Assay

Antiviral activity can be quantitatively measured using a multinuclear activation of a galactosidase indicator (MAGI) assay. See, Kimpton and Emerman (1992) J Virol. 66:2232. Target cells (e.g., a mammalian cell, for example, HeLa cells) that have been stably transfected with a target cell surface viral receptor (e.g., CD4, a chemokine receptor) and a reporter construct that expresses a beta-galactosidase protein modified to localize to the nucleus under the control of viral transcription elements (e.g., HIV-1 LTR and HIV-1 Tat). To drive expression of the modified beta-galactosidase protein, infecting virus must produce viral transcription elements (e.g., HIV-1 Tat). The nuclei of target cells that have been infected and induced to express the modified beta-galactosidase protein will stain blue, and this can be easily visualized using a light microscope (e.g., 100× magnification). The MAGI assay is usually read on day 2 or day 3 after initiation of infection. The number of blue cells indicates the number of infectious virus particles in the inoculum. The blue nuclei can be counted using the light microscope. A 50% inhibitory concentration (IC₅₀) is then determined from a plot of % inhibition vs. log₁₀ drug concentration.

Synergistic antiviral effects of thiophosphonoformic acid and one or more nucleotide/nucleoside reverse transcriptase inhibitors can be determined using the median effect principle (see, Chou and Talalay, 1984, supra, Chou and Rideout, 1996, supra, and Reynolds and Maurer, supra) and quantitatively determined by combination indices (CI). A combination index value less than 1.0 indicates synergism between the active agents. Combination index values can be further graded as follows:

| Combination Index Value | Level of Synergy |
|---|---|
| 0.9-1.0 | slight |
| 0.7-0.9 | moderate |
| 0.3-0.7 | average |
| 0.1-0.3 | strong |
| <0.1 | very strong |

ii. PhenoSense™ Assay

Antiviral activity also can be quantitatively measured using a PhenoSense™ assay. A PhenoSense™ assay uses nucleic acid amplification to derive HIV protease and reverse transcriptase nucleic acid sequences from plasma. The patient-derived sequences are incorporated into a viral vector to construct a resistance test vector (RTV). The viral vector also contains an indicator or reporter gene (e.g., green fluorescent protein, luciferase, beta galactosidase, etc.) inserted within a deleted portion of the HIV envelope gene. The reporter gene is under the control of a strong promoter (e.g., a CMV promoter) in the RTV.

The PhenoSense™ assay is performed by introducing the RTV nucleic acid sequence into host cells, collecting virus particles after transfection, and using the virus particles to infect target cells. Drug susceptibility is measured by comparing reporter gene activity produced in the presence and absence of drugs. The presence of drug can be one or more different concentrations, for example, several titrated concentrations.

The data are analyzed by plotting the percent inhibition of viral replication, as measured by reporter gene activity, against the log₁₀ concentration of drug for the patient virus (in the RTV) and a drug sensitive reference virus. The resulting drug susceptibility curve is used to calculate the concentration of drug required to inhibit viral replication by 50% (IC₅₀). A shift in the patient inhibition curve toward a higher drug concentration as compared to the curve of the drug-sensitive reference virus is interpreted as reduced drug susceptibility. A shift in the patient inhibition curve toward a lower drug concentration as compared to the curve of the drug-sensitive reference virus is interpreted as increased drug susceptibility. PhenoSense™ can be conducted by a commercial service provider, for example, Monogram Biosciences, South San Francisco, Calif.

Synergism of one or more active agents can be calculated using Combination Index Values, as described above.

iii. p24 Detection Assay

Reduction in viral titer also can be measured using an ELISA capture assay with antibodies specific for viral capsid proteins (e.g., HIV-1 p24). ELISA kits for measuring HIV-1 p24 are commercially available from, for example, Zeptometrix, Buffalo, N.Y.; PerkinElmer, Wellesley, Mass.; and Aalto Bio Reagents, Dublin, Ireland.

b. In Vivo

Synergistic viral load reduction by combinations of thiophosphonoformic acid and one or more nucleoside/nucleotide reverse transcriptase inhibitors also can be measured in vivo. Virus titers can be detected from samples from a patient (e.g., blood, serum, plasma) and compared to a reference sample, for example, an sample taken at an earlier time point from the same patient.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

The following examples are offered to illustrate, but not to limit the claimed invention.

### Materials and Methods

The following materials and methods were used in the examples provided herein.

### Cell Lines and Virus

MAGI cells were maintained in DMEM medium containing 10% fetal calf serum and 5 mM glutamine. H9 cells were maintained in RPMI 1640 medium containing 10% fetal calf serum. HIV-1_{IIIB} and HIV-1_{LAI} were obtained through the AIDS Research and Reference Reagent Program, Division of AIDS, NIAID, NIH. HIV-1_{LAI} was originally contributed by Dr. Jean-Marie Bechet and Dr. Luc Montagnier, courtesy of the MRC AIDS Directed Programme. HTLV-IIIB/H9 was received from Dr. Robert Gallo. HIV-1_{IIIB}, HIV-1_{HXB2}, and HIV-1_{LAI} virus stocks were prepared from acutely infected H9 cells.

### Drugs

Thiophosphonoformic acid (Thiovir™) was produced at deCODE genetics (Lemont, Ill.). Foscaret was obtained from Sigma-Aldrch (St. Louis, Mo.). Azidothymidine (“AZT”) or Zidovudine (“Retrovir”) I.V. Infusion was obtained from G1axo Welcome (Research Triangle Park, N.C.). Nevirapine obtained through the AIDS Research and Reference Reagent Program, NIAID, NIH: Nevirapine.

### IC₅₀ Determination

Wild type and antiviral drug resistant HIV were tested for sensitivity to anti-viral drugs using either MAGI assay or Phenosense™ assay. The 50% inhibitory concentration, IC₅₀, was then determined from a plot of percent inhibition vs. log₁₀ drug concentration.

### MAGI Assay

MAGI cells were seeded at approximately 5×10⁴ cells per well in a 12 well plate in DMEM with 10% fetal calf serum and incubated overnight at 37° C. in 5% CO₂. Drug was added at various concentrations and incubated for approximately 30 to 60 minutes. Cells were infected with either HIV-1 HXB2 or HIV-1_{IIIB} in the presence of either 4 microg/ml polybrene or 60 microg/ml dextran. Infection was continued for 48 hours and cells fixed using 0.05% glutaraldehyde. Cells were washed three times with PBS and stained with 4 mM potassium ferrocyanide, 4 mM potassium ferricyanide, 2 mM MgCl₂ and 0.8 mg/ml X-gal. Blue nuclei were counted under a microscope at 100× magnification.

### H9 Infection and p24 Antigen Capture Assay

H9 T-cells were pretreated with polybrene at 4 μg/ml and drug at indicated concentrations in RPMI plus 10% fetal calf serum for 30 minutes prior to adsorption of virus at multiplicities of infection (MOI) 0.1 for 2 hours at 37° C., 5% CO₂. Cells were washed 3 times with PBS and resuspended in fresh media plus drug. Incubation at 37° C., 5% CO₂ was continued for 4-6 days and cells centrifuged at 1200 rpm. Supernatants were removed and p24 detected by quantitative antigen capture kit (Zeptometrix, Buffalo, N.Y.).

### Phenosense™ Assay

All Phenosense™ assays were performed at Virologic, Inc as described (Petropoulos, et al. (2000) Antimicrob Agents Chemother 44:920). Briefly, the envelope gene is deleted from wild type HIV-1 and luciferase reporter inserted under control of a strong CMV promoter. Viral stocks are produced by co-transfection of producer cells with the recombinant viral DNA and a vector that expresses envelope protein from an amphotropic murine leukemia virus. Pseudotyped viral particles are replication defective and only undergo one round of infection. Expression of the luciferase gene in target cells is dependent on a single round of infection.

### Selection of Drug Resistant Variants

To select for drug resistant variants, 1×10⁶H9 cells were pretreated for two hours at three concentrations of AZT (0.5, 1 and 2 μM), Nevirapine (0.5, 1 and 2 μM), foscarnet (25 and 50 μM) and Thiovir™ (25 and 50 μM) prior to infection at MOI of 0.1 of HIV_{IIIB} or HIV_{LAI}. Cultures with the highest level of drug that showed cytopathic effect and at least 100-fold increase in p24 level at 7 days post-infection where harvested and supernatants used to infect fresh H9 cells at twice the drug concentration. Virus was passaged at increasing drug concentration and monitored for increase in IC₅₀. Reverse transcriptase gene was PCR amplified from H9 cells infected with resistant virus using primers 881For′ (AATTAACCCTCACTAAAGGGAGACAGAGCCAACAGCCCCACCA) and 929T7Rev′ (ATTTAATACGACTCACTATAGGGATTTCCCCACTAACTTCTGATGTCATTGACA) containing T7 sequences. The reverse transcriptase gene was sequenced from the purified PCR product using universal 17 primer and compared to virus passaged in the absence of drug and published sequence.

### Example 1

### Activities of Thiovir™ Against NRTI and NNRTI Resistant Virus

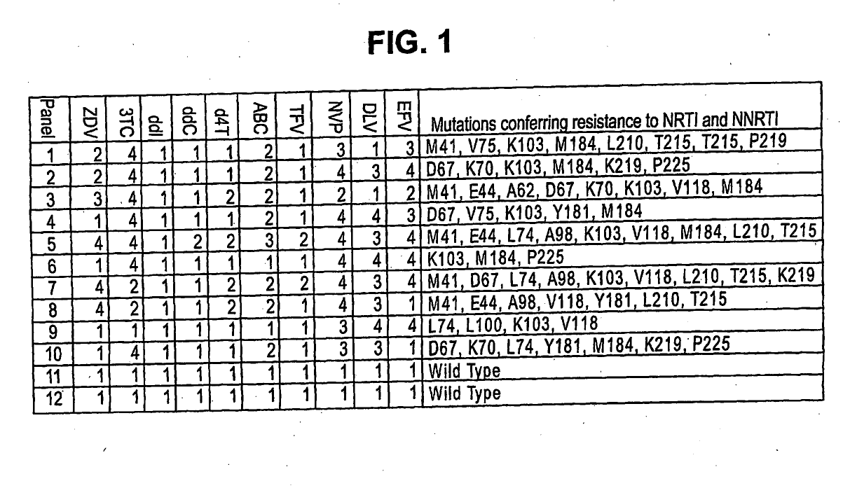

The activity of Thiovir™ was evaluated against viruses that are resistant to common NRTI and NNRTI drugs using the Phenosense™ reporter system (Petropoulos, supra). A panel of ten viruses with multiple mutations conferring varying resistance to NRTI and NNRTI was examined (FIG. 1). In addition, two wild type viruses were included in the viral panel. Each virus was tested against Thiovir™, foscarnet, two NRTI (stavudine and zidovudine), and two NNRTI (efavirenz and nevirapine).

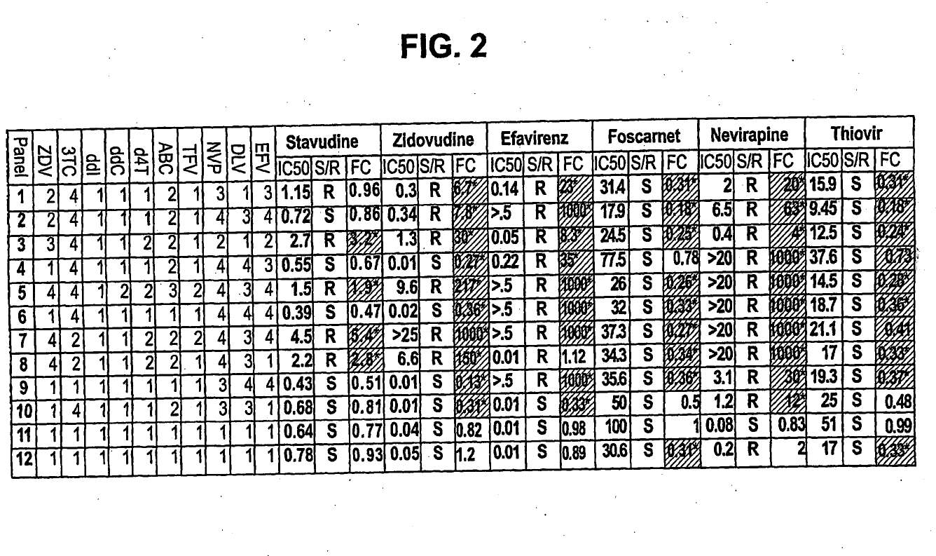

Thiovir™ and foscarnet showed similar IC₅₀ values against the wild type virus (FIG. 2). IC₅₀ values for mutant virus were compared to wild type virus IC₅₀ values to determine the fold change in resistance or sensitivity. The clinical cutoff value (FIG. 2, light grey highlighted values) is defined as the level at which the probability of failure of therapy significantly increases in resistant patients. Approximately half the variant virus are resistant to the NRTI tested and nearly all are resistant to the NNRTI tested. Most resistant virus is above the clinical cutoff in both NRTI and NNRTI. In contrast, all viruses Were sensitive to both foscarnet and Thiovir™, the majority being hypersensitive (FIG. 2, dark grey highlighted values). The data is consistent with the conclusion that Thiovir™ is effective against virus that is resistant to both NRTI and NNRTI.

### Example 2

### Synergistic Inhibition of HIV by Thiovir™ and Zidovudine (AZT)—MAGI Assay

Previous studies have shown slight to moderate synergy between foscarnet and zidovudine at ratios ranging from 1:300 to 1:3000 (Hostetler, et al., (2000) Antivir Chem Chemother 11:213). We investigated combinations of Thiovir™ or foscarnet and zidovudine by MAGI assay (Klimpton and Emerman, supra). Drug interactions were determined using the median effect principle (Chou and Talalay (1984) Adv Enzyme Regul 22:27). Combination indices (CI) quantify synergy, summation and antagonism as follows: CI<1 (synergy); CI=1 (summation); CI>1 (antagonism). Since dose-effect relationships of Thiovir™ or foscarnet with zidovudine are not parallel in the median effect plot, exclusivity of effects cannot be established and data for both mutually exclusive (i.e., competitive inhibitors) and mutually nonexclusive (i.e., noncompetitive inhibitors) assumptions are shown.

Average combination indices are approximately 0.55 for zidovudine plus Thiovir™ mixed at a ratio of 1:1 indicating strong synergism (Table 1). In contrast, zidovudine plus foscarnet indices range from only slightly synergistic to antagonistic, with average combination indices of approximately 1.5. The strong synergy between zidovudine and Thiovir™ implies antiviral efficacy can be increased, even at reduced drug dosage, allowing for suitable drug regimens combining Thiovir™ with an NRTI.

TABLE 1
Combination Index Values for Zidovudine Combined With
Thiovir ™ or Foscarnet (MAGI Assay)

| | | Combination | | | |
|---|---|---|---|---|---|
| Drug Combination | | Index Values | | | |
| (molar ratio) | Model | IC₅₀ | IC₇₅ | IC₉₀ | r² Value |
| Zidovudine:Thiovir ™ | Mutually | 0.39 | 0.51 | 0.68 | 0.97 |
| (1:1) | Exclusive | | | | |
| | Mutually | 0.39 | 0.52 | 0.68 | |
| | Nonexclusive | | | | |
| Zidovudine:Thiovir ™ | Mutually | 0.57 | 0.5 | 0.44 | 0.96 |
| (1:10) | Exclusive | | | | |
| | Mutually | 0.60 | 0.53 | 0.47 | |
| | Nonexclusive | | | | |
| Zidovudine:Thiovir ™ | Mutually | 0.85 | 0.75 | 0.67 | 0.97 |
| (1:100) | Exclusive | | | | |
| | Mutually | 10.4 | 0.88 | 0.77 | |
| | Nonexclusive | | | | |
| Zidovudine:Foscarnet | Mutually | 1.65 | 1.49 | 1.55 | 0.97 |
| (1:1) | Exclusive | | | | |
| | Mutually | 1.70 | 1.55 | 1.42 | |
| | Nonexclusive | | | | |
| Zidovudine:Foscarnet | Mutually | 1.01 | 0.78 | 0.61 | 0.93 |
| (1:10) | Exclusive | | | | |
| | Mutually | 1.14 | 0.88 | 0.68 | |
| | Nonexclusive | | | | |

### Example 3

### Synergistic Inhibition of HIV by Thiovir™ and Zidovudine (AZT)—p24 Assay

Synergy between Thiovir™ and zidovudine was further investigated by infection of H9 T-cells in the presence of a fixed ratio of drug and analysis by the p24 assay (Table 2). Similar to the MAGI assay, combination indices indicate strong synergy of Thiovir™ combined with zidovudine at ratios of 1:1 and 1:10. In contrast to Thiovir™, combination of foscarnet and zidovudine results in antagonism at a 1:1 ratio and synergy at a 1:10 ratio. The data is consistent with the conclusion that Thiovir™ has a lower effective dose than foscarnet in combination therapy with zidovudine.

TABLE 2
Combination Index Values for Zidovudine Combined With
Thiovir ™ or Foscarnet (p24 Assay)

| | | Combination | | | |
|---|---|---|---|---|---|
| Drug Combination | | Index Values | | | |
| (molar ratio) | Model | IC₅₀ | IC₇₅ | IC₉₀ | r² Value |
| Zidovudine:Thiovir ™ | Mutually | 0.55 | 0.85 | 0.92 | 0.83 |
| (1:1) | Exclusive | | | | |
| | Mutually | 0.56 | 0.86 | 0.89 | |
| | Nonexclusive | | | | |
| Zidovudine:Thiovir ™ | Mutually | 0.43 | 0.46 | 0.52 | 0.96 |
| (1:10) | Exclusive | | | | |
| | Mutually | 0.45 | 0.48 | 0.53 | |
| | Nonexclusive | | | | |
| Zidovudine:Foscarnet | Mutually | 1.63 | 2.58 | 4.10 | 0.90 |
| (1:1) | Exclusive | | | | |
| | Mutually | 1.65 | 2.63 | 4.19 | |
| | Nonexclusive | | | | |
| Zidovudine:Foscarnet | Mutually | 0.44 | 0.46 | 0.47 | 0.98 |
| (1:10) | Exclusive | | | | |
| | Mutually | 0.46 | 0.47 | 0.48 | |
| | Nonexclusive | | | | |

### Example 4

### Synergistic Inhibition of HIV by Thiovir™ and Tenofovir—p24 Assay

Tenofovir is an acyclic phosphonate analogue of a nucleotide reverse transcriptase inhibitor (NtRTI) that is widely used as a replacement or complement to an NRTI in combination HIV therapy. Synergy between Thiovir and a tenofovir was investigated by the p24 assay. Thiovir showed synergy at a ratio of 1:1 with tenofovir. In contrast, foscarnet showed additive to antagonistic behavior when combined with tenofovir at the same ratio (Table 3). The data is consistent with the conclusion that Thiovir has a lower effective dose than foscarnet in combination therapy with tenofovir.

TABLE 3
Combination Index Values for Tenofovir Combined With
Thiovir ™ or Foscarnet (p24 Assay)

| | | Combination | | | |
|---|---|---|---|---|---|
| Drug Combination | | Index Values | | | |
| (molar ratio) | Model | IC₅₀ | IC₇₅ | IC₉₀ | r² Value |
| Tenofovir:Thovir ™ | Mutually | 0.86 | 0.88 | 0.91 | 0.98 |
| (1:1) | Exclusive | | | | |
| | Mutually | 0.86 | 0.89 | 0.93 | |
| | Nonexclusive | | | | |
| Tenofovir:Thiovir ™ | Mutually | 1.04 | 0.97 | 0.92 | 0.97 |
| (1:10) | Exclusive | | | | |
| | Mutually | 1.10 | 1.05 | 1.01 | |
| | Nonexclusive | | | | |
| Tenofovir:Foscarnet | Mutually | 1.61 | 1.25 | 0.95 | 0.98 |
| (1:1) | Exclusive | | | | |
| | Mutually | 1.65 | 1.26 | 0.96 | |
| | Nonexclusive | | | | |

### Example 5

### Resistance Profile of Wild Type HIV to Thiovir

Thiovir™ and foscarnet resistant viruses were selected by in vitro passage of HIV_{LAI} on H9 cells in the presence of escalating concentrations of drug. Resistant virus was evaluated for phenotypic changes by IC₅₀ determination using the MAGI assay. Furthermore, the viral DNA was sequenced to determine genotypic variations (Table 4). Two independent 13 to 15 round selections were performed.

TABLE 4
Drug Susceptibility and Genetic Mutations of Thiovir ™
and Foscarnet Resistant HIV

| Drug | | | | | | |
|---|---|---|---|---|---|---|
| Resistant | Susceptibility Index | | | | | |
| Virus | (IC_{50 Resistant Virus}/IC_{50 Purent Virus}) | | | | | Amino Acid |
| (Selection #) | Thiovir ™ | Foscarnet | Zidovudine | Nevirapine | Codon Changes | Changes |
| Thiovir ™ | 4.8 | 4.8 | 0.22 | 1.05 | CTT → TTT | L214F |
| (1) | | | | | AGA → AAA | R172K |
| | | | | | CAA → CGA | Q174R |
| Thiovir ™ | 113 | 85 | 0.35 | N/A | CTT → TTT | L214F |
| (2) | | | | | AUG → AUA | M184I |
| Foscarnet | 6.3 | 4.1 | 0.87 | 1.06 | CTT → TTT | L214F |
| (1) | | | | | AGA → AAA | R172K |
| Foscarnet | 100 | 103 | 0.58 | N/A | CTT → TTT | L214F |
| (2) | | | | | AUG → AUA | M184I |

Two resistant viruses, exhibiting 4.8 fold and 85 fold resistance to Thiovir™ were selected. Virus resistant to Thiovir™ also showed a similar resistance to foscarnet, indicating cross resistance. Furthermore, virus selected for foscarnet resistance had a similar resistance to Thiovir™, again indicating cross resistance. Consistent with previous data, both Thiovir™ and foscarnet selected virus were sensitive to nevirapine and hypersensitive to zidovudine. Prolonged in vitro selection of wild-type or AZT-resistant HIV-1 strains with the combination AZT and Thiovir™, or AZT and foscarnet, failed to generate co-resistant virus, suggesting Thiovir™ and AZT resistance profiles are mutually exclusive.

Both Thiovir™ and foscarnet resistant viruses contained the variations L214F and R172K for selection 1. An additional mutation, Q174R, was present only in the Thiovir™ resistant virus. For selection 2, both Thiovir™ and foscarnet contained L214F and M184I mutations. L214F and M184I have been previously associated with foscarnet and foscarnet prodrug resistance (Hammond, et al., (2001) Antimicrob Agents Chemother 45:1621). R172K and Q174R are novel mutations that have not been previously linked to resistance to foscarnet or any foscarnet derivatives. The results are consistent with the conclusion that the Thiovir™ resistance profile is similar to foscarnet. The mutation profile of Thiovir™ and foscarnet resistant viruses are consistent with suppression of the zidovudine resistance phenotype.

In conclusion, the Examples demonstrate the synergistic activities of combinations of thiophosphonoformic acid and an NRTI, allowing for reduced dosages of one or both active agents.

What is claimed is:

1. A method of reducing viral load in a patient infected with a virus, the method comprising the oral co-administering to the patient of an amount of thiophosphonoformic acid and an amount of a nucleoside or nucleotide reverse transcriptase inhibitor where the amounts being co-administered are sufficient to synergistically lower the viral load and where the thiophosphonoformic acid is orally administered in a dosage range of between 2 mg/kg to 50 mg/kg per day.

2. The method of claim 1, wherein the dosage of thiophosphonoformic acid is administered in a dosage range of between 15 mg/kg to 40 mg/kg per day.

3. The method of claim 1, wherein the dosage of thiophosphonoformic acid is administered in a dosage range of between 20 mg/kg to 35 mg/kg per day.

4. The method of claim 1, wherein the dosage of the nucleoside or nucleotide reverse transcriptase inhibitor is reduced by at least 10-fold in comparison to when the nucleoside or nucleotide reverse transcriptase inhibitor is administered without thiophosphonoformic acid.

5. The method of claim 1 wherein the nucleoside reverse transcriptase inhibitor is selected from the group consisting of: zidovudine, didanosine, stavudine, lamivudine, abacavir and emtricitabine.

6. The method of claim 5 wherein the nucleoside reverse transcriptase inhibitor is zidovudine.

7. The method of claim 1 where the nucleotide reverse transcriptase inhibitor is tenofovir.

8. The method of claim 1 where the virus is a retrovirus.

9. The method of claim 8 where the retrovirus is Human Immunodeficiency Virus (HIV).

10. The method of claim 1 where the virus is a herpes virus.

11. The method of claim 1 where the thiophosphonoformic acid and the nucleoside or nucleotide reverse transcriptase inhibitor are simultaneously administered in a combined formulation.

12. The method of claim 1 where the thiophosphonoformic acid and the nucleoside or nucleotide reverse transcriptase inhibitor are simultaneously administered in a combined formulation tablet.

13. An oral medicament for lowering viral load in a patient infected with a virus, where the medicament is a combined formulation comprising an amount of thiophosphonoformic acid and an amount of a nucleoside or nucleotide reverse transcriptase inhibitor where the thiophosphonoformic acid and nucleoside or nucleotide reverse transcriptase inhibitor are present in amounts sufficient to synergistically lower viral load and the medicament contains between 150 mg and 750 mg of thiophosphonoformic acid per combined formulation.

14. The medicament of claim 13 where the nucleoside reverse transcriptase inhibitor is zidovudine.

15. The medicament of claim 13 where the thiophosphonoformic acid and nucleoside or nucleotide reverse transcriptase inhibitor are in a capsule.

16. The medicament of claim 13 where the thiophosphonoformic acid and nucleoside or nucleotide reverse transcriptase inhibitor are in a tablet.

17. The medicament of claim 13 where the thiophosphonoformic acid and nucleoside or nucleotide reverse transcriptase inhibitor are in a fluid.

18. The medicament of claim 13 where the virus is a retrovirus.

19. The medicament of claim 18 where the retrovirus is Human Immunodeficiency Virus (HIV).

20. The medicament of claim 13 where the virus is a herpes virus.

21. The use of thiophosphonoformic acid and a nucleoside or nucleotide reverse transcriptase inhibitor in the manufacture of a medicament for the therapeutic and/or prophylactic treatment of a viral infection where the thiophosphonoformic acid and nucleoside or nucleotide reverse transcriptase inhibitor are present in amounts sufficient to synergistically lower viral load and the medicament contains between 150 mg and 750 mg of thiophosphonoformic acid per combined formulation.

22. The use of thiophosphonoformic acid and a nucleoside or nucleotide reverse transcriptase inhibitor in the preparation of an anti-viral agent in ready-to-use drug form for treating or preventing virus infections where the where the thiophosphonoformic acid and nucleoside or nucleotide reverse transcriptase inhibitor are present in amounts sufficient to synergistically lower viral load and the ready-to-use drug form contains less than 500 mg of thiophosphonoformic acid per form.
